(19) United States
(12) Patent Application Publication
(10) Pub. No.: US 2017/0219558 A1
(43) Pub. Date: Aug. 3, 2017

(54) BIOCHEMICAL DETECTION APPARATUS

(71) Applicant: Ming-Jui LI, Tainan City (TW)

(72) Inventor: Ming-Jui LI, Tainan City (TW)

(21) Appl. No.: 15/014,678

(22) Filed: Feb. 3, 2016

(51) Int. Cl. G01N 33/487; H04N 5/232

(52) U.S. Cl. CPC G01N 33/48792; H04N 5/232

(57) ABSTRACT

A biochemical detection apparatus includes an electronic computing device and an optical device. The optical device includes a main body. The main body includes an accommodating space provided in the main body, and an insertion slot, an optical lens and an opening that are in communication with the accommodating space. The optical lens and the opening are disposed on a same plane of the main body, and the optical lens is located above an image capturing unit of the electronic computing device. The optical device may be installed at a handheld electronic computing device commonly carried by an individual, and is capable of immediately performing detection for an environmental parameter or a biological parameter by a biochemical sheet. The optical device having a simple structure is small in volume, convenient and low in cost, and may replace expensive precision detection apparatuses.

### FIELD OF THE INVENTION

The present invention relates to a detection apparatus, and particularly to a biochemical detection apparatus. The biochemical detection apparatus is capable of performing detection for an environmental parameter or a biological parameter by a biochemical sheet and an electronic computing device without time and location limitations.

### BACKGROUND OF THE INVENTION

In the early years, when detection for an environmental parameter such as the amount of a metal component of a contaminant or a pH value in the environment, or for a biological parameter such as blood sugar in the human body, is performed by a biochemical sheet, the object under test, i.e., the contaminant or blood sugar, is mixed with a reactant on the biochemical sheet to produce a change. A precision apparatus or manual means is the applied to identify or determine whether the object under test satisfies a standard.

The above manual means for detecting an object under test is time saving. However, the change after a reaction of the object under test may vary due to the observation and determination of different individuals, leading to unstable outcomes that cannot be relied upon as a reference standard in the subsequent detection. Further, conventional biochemical sheets used for specific detection are more costly. Once the number of times of detection gets large, the amount of biochemical sheets used is increased to inevitably increase investment costs of a business entrepreneur.

Therefore, a method of an optical image extending apparatus is proposed to improve the determination method performed by manual means. With an optical lens disposed, an image capturing unit captures an image of a change result in a biochemical sheet, and the image is further extended and determined. The accuracy and efficiency of such method are higher than those of the determination method by manual means.

However, current commercially available optical image extending apparatuses not only have sophisticated and complex components and higher costs, but also have an excessively large overall volume such that they are not readily portable and hence quite inconvenient. Thus, these optical image extending apparatuses may not be applied to immediately detect an object under test, such as the amount of a metal component or a pH value of the environment or a biological parameter of the human body. Therefore, there is a need for a solution that solves the drawbacks of high costs and the incapability of immediate detection due to poor portability of a conventional optical image extending apparatus.

### SUMMARY OF THE INVENTION

It is an object of the present invention to provide a biochemical detection apparatus incorporating an electronic computing device and a low-cost and readily portable optical device having a small volume. Thus, the biochemical detection apparatus of the present invention is capable of immediately detecting an environmental parameter such as the amount of a metal component of a contaminant in the environment or a pH value, or a biological parameter such as blood sugar in the human body.

According to the above object, the present invention provides a biochemical detection apparatus including an electronic computing device and an optical device. The electronic computing device includes at least one display unit, and at least one image capturing unit coplanar with the display unit. The optical device includes a main body. The main body includes an accommodating device provided in the main body, an insertion slot in communication with the accommodating space, an optical lens in communication with the accommodating space, and an opening in communication with the accommodating space. The optical lens and the opening are disposed on a same plane of the main body, and the optical lens is located above the image capturing unit.

The biochemical detection apparatus further includes a biochemical sheet inserted in the insertion slot. The biochemical sheet includes a test area and a calibration area disposed at one end thereof. The test area and the calibration area are located above the image capturing unit.

The present invention provides features below.

1. In the present invention, the optical device is a readily portable and low-cost structure having a small volume. Thus, a user may immediately apply the present invention to a fluid or a gas in the environment to detect the amount of a metal component of a contaminant, a pH value, or the presence of contamination, or to detect an object under test such as a biological fluid in the human body, including blood, urine or saliva to conveniently monitor the level of a special an analyte (e.g., glucose, cholesterol, ketone or a specific protein) existing in the fluid. To put to application, an object under test is added to the test area of the biochemical sheet to allow the object under test to mix with a biochemical reactant on the test area. The biochemical sheet is then inserted into the insertion slot to cause the test area to align with the optical lens, so as to allow the image capturing unit to scan a change result of the test area, and to perform image extension and determination. The detection result is displayed on the display unit immediately for the user to determine detection data of the object under test, thereby solving the drawbacks of high costs and the incapability of immediate detection due to poor portability of an expensive conventional apparatus.

2. Through an application program, the electronic computing device of the present invention divides a part of the display unit into a light emitting area, and locates the light emitting area below the opening of the main body to have the illumination from the light emitting area serve as a main light source for the image capturing unit to capture an image of the test area. The light beams of the light emitting area further irradiate into the accommodating space through the opening to provide the image capturing unit with sufficient light beams for scanning and facilitating the observation of the change result of the test area.

3. Through the application program, the electronic computing device of the present invention simultaneously divides the display unit into the light emitting area and a display area. Thus, the user is allowed to at the same time observe the change result of the test area through the display area while the image capturing unit scans the change result of the test area.

4. In the present invention, one end of the biochemical sheet is simultaneously embedded with the test area and the calibration area. Thus, the user may directly compare the change result of the test area with a comparison reference object at the calibration area to determine the difference between the change result and the comparison reference object, which is distinct from the drawback of the test area and the calibration area belonging to two different objects and being more costly and inconvenient in a conventional solution.

5. When detection is performed using the biochemical sheet, the detection result can be manually observed. Alternatively, the image capturing unit is controlled through the application program to cause the image capturing unit to automatically scan the change result of the test area and the comparison reference object. A difference between the change result of the test area and the comparison reference object is then automatically calculated, and the calculated result is displayed on the display area.

6. In the present invention, a focusing target object is embedded in the calibration area to allow the image capturing unit to perform preceding operations including focusing, image alignment and light source adjustment to enhance the detection accuracy.

7. The electronic computing device of the present invention may store the calculated result to a database to serve for data statistics, data analysis and subsequent remote support applications.

8. In the present invention, with the optical lens disposed between the test area and the image capturing unit, the image capturing unit is allowed to scan the change result of the test area through the properties of the optical lens to provide image extension. Thus, the test area is capable of detecting the object under test with the biochemical reactant in a quite small area, thereby reducing the area that the test area requires and reducing the object amount under test, increasing the amount of detection and reducing investment costs of the business entrepreneur.

### BRIEF DESCRIPTION OF THE DRAWINGS

FIG. 1 is an exploded perspective view of an electronic computing device and an optical device of the present invention;

FIG. 2 is an exploded perspective view of an optical device and a biochemical sheet of the present invention;

FIG. 3 is a perspective sectional view of an optical device and a biochemical sheet of the present invention;

FIG. 4A is a planar view of an assembly of an electronic computing device, an optical device and a biochemical sheet of the present invention;

FIG. 4B is a sectional view of FIG. 4A along 4B-4B;

FIG. 5 is a schematic diagram of a biochemical detection apparatus of the present invention in another application form;

FIG. 6 is a planar view of a fixing member according to a first embodiment of the present invention;

FIG. 7 is a perspective view of a fixing member according to a second embodiment of the present invention; and

FIG. 8 is a perspective view of an electronic computing device in another form and an optical device of the present invention.

### DETAILED DESCRIPTION OF THE PREFERRED EMBODIMENTS

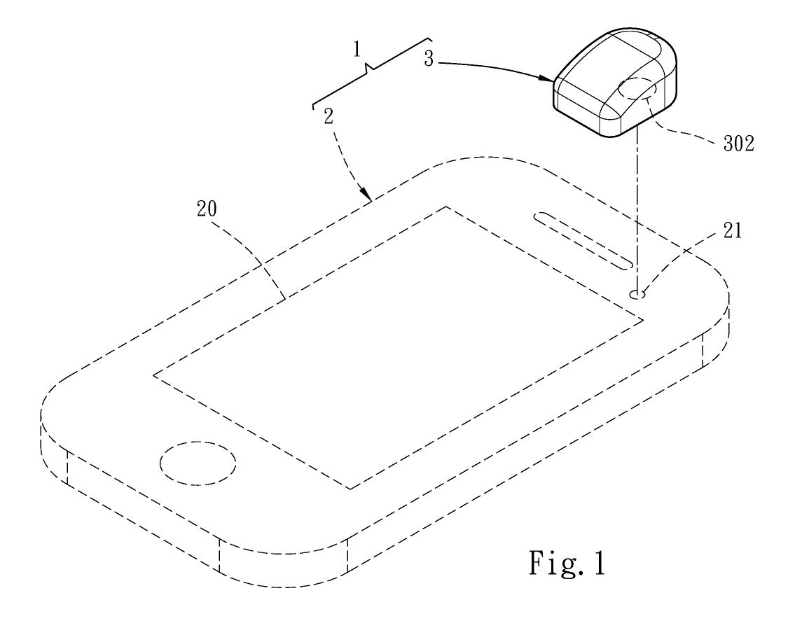

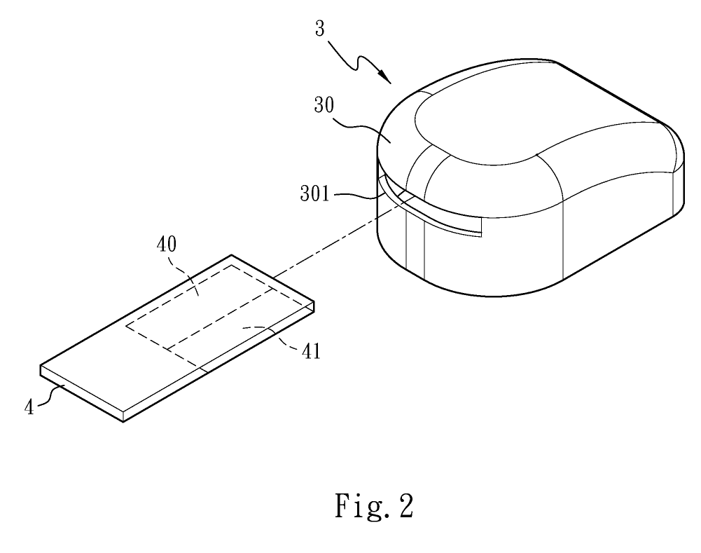

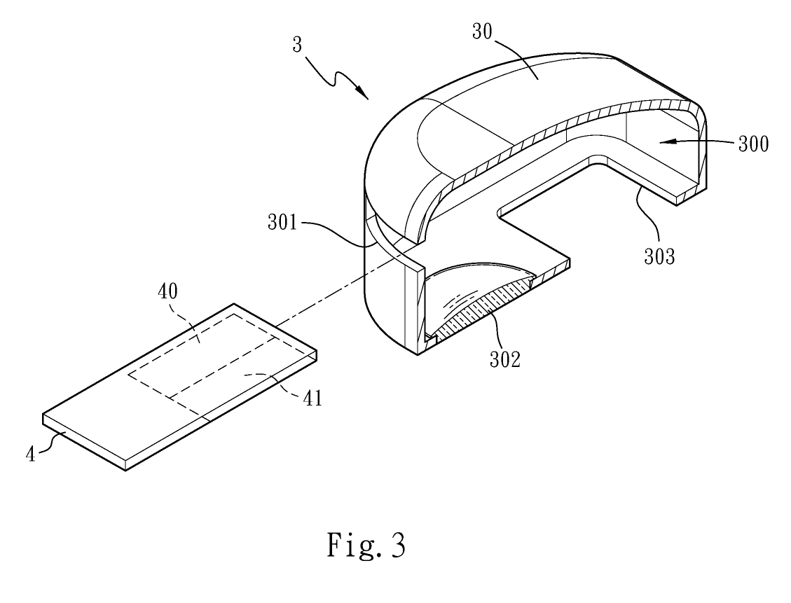

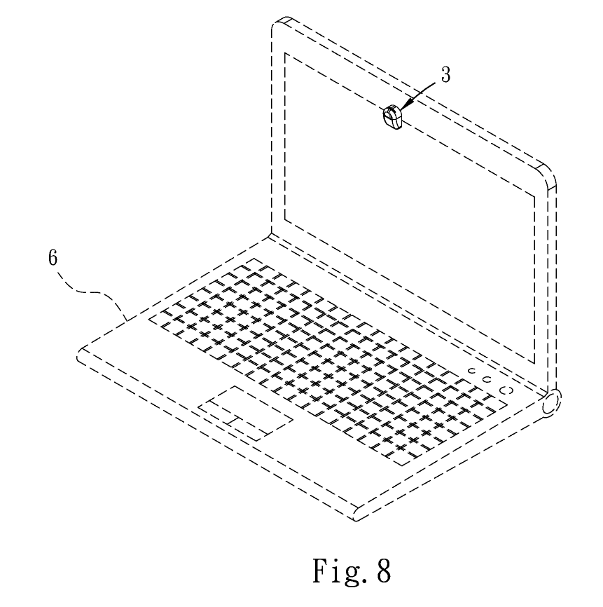

Referring to FIG. 1 to FIG. 3 showing a biochemical detection apparatus 1 of the present invention, the biochemical detection apparatus 1 includes an electronic computing device 2 and an optical device 3. The electronic computing device 2 includes at least one display unit 20, and at least one image capturing unit 21 coplanar with the display unit 20. The optical device 3 includes a main body 30, which includes an accommodating space 300 provided in the main body 30, an insertion slot 301 in communication with the accommodating space 300, an optical lens 302 in communication with the accommodating space 300, and an opening 303 in communication with the accommodating space 300. The optical lens 302 and the opening 303 are disposed on a same plane of the main body 30, and the optical lens 302 is located above the image capturing unit 21. In an embodiment of the present invention, the electronic computing device 2 may a handheld smart mobile device with a computing function, or may be an electronic computing device such as a laptop computer 6 or a tablet computer shown in FIG. 8. Further, the display unit 20 and the image capturing unit 21 are located at a same front side. More specifically, the display unit 20 and the image capturing unit 21 may be implemented as a screen and a lens at a front side, respectively. Alternatively, the display unit 20 and the image capturing unit 21 may be implemented as a screen and a lens at a rear side, respectively. In the embodiment of the present invention, for example but not limited to, the optical lens 302 is a convex lens.

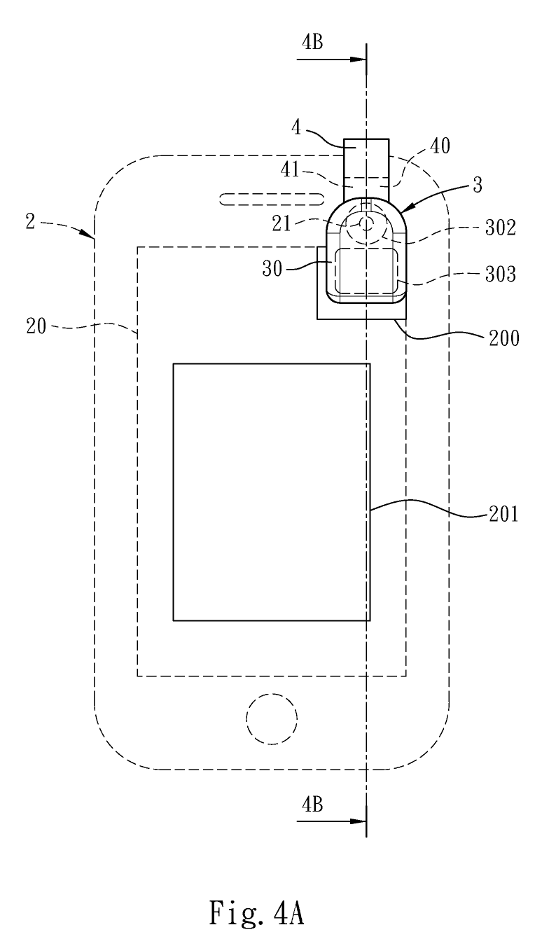

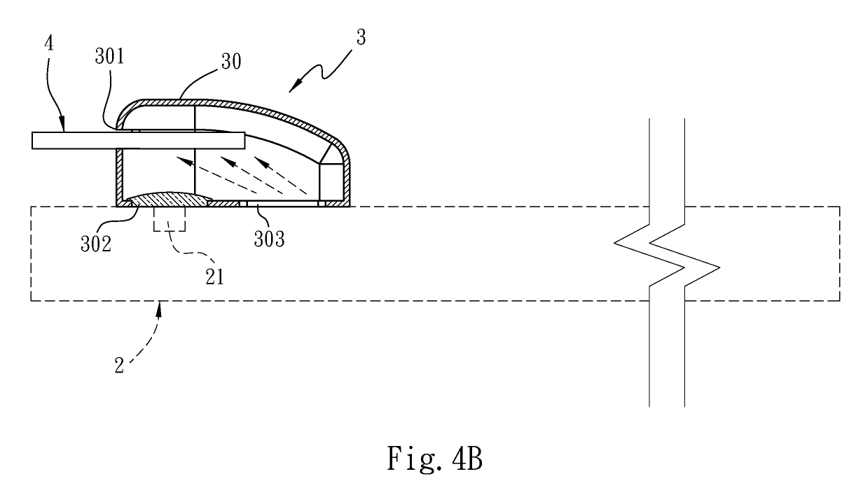

Referring to FIG. 4A and FIG. 4B, the biochemical detection apparatus 1 of the present invention further includes a biochemical sheet 4. The biochemical sheet 4, inserted in the insertion slot 301 of the main body 30, includes a test area 40 and a calibration area 41 at one end thereof. The test area 40 and the calibration area 41 are accommodated in the accommodating space 300, and are located above the image capturing unit 21 for the image capturing unit 21 to scan. The optical device 3 of the present invention is a readily portable structure having a small volume and a low cost. To put to application, an object under test is added to the test area 40 of the biochemical sheet 4 to allow the object under test to mix with a biochemical reactant on the test area 40. The biochemical sheet 4 is then inserted into the insertion slot 301 to cause the test area 40 to align with the optical lens 302, so as to allow the image capturing unit 21 to scan a change result of the test area 40, and to perform image extension and a determination. The detection result is displayed on the display unit 20 immediately for the user to determine detection data of the object under test, thereby solving the drawbacks of high costs and the incapability of immediate detection due to poor portability of an expensive conventional optical image extending apparatus. The biochemical reactant on the test area 40 of the present invention is applicable to a fluid or a gas in the environment to detect the amount of a metal component of a contaminant, a pH value, or the presence of contamination, or to detect an object under test, e.g., blood sugar in a biological fluid including blood, urine saliva of the human body to conveniently monitor the level of a special an analyte (e.g., glucose, cholesterol, ketone or a specific protein) existing in the fluid.

In the present invention, the optical lens 302 is disposed between the test area 40 and the image capturing unit 21. Thus, the image capturing unit 21 provides image extension through the properties of the optical lens 302, such that the test area 40 is allowed to detect the object under test using the biochemical reactant having a quite small area, thereby reducing the area that the test area requires and reducing the object amount under test , increasing the amount of detection and reducing investment costs of a business entrepreneur.

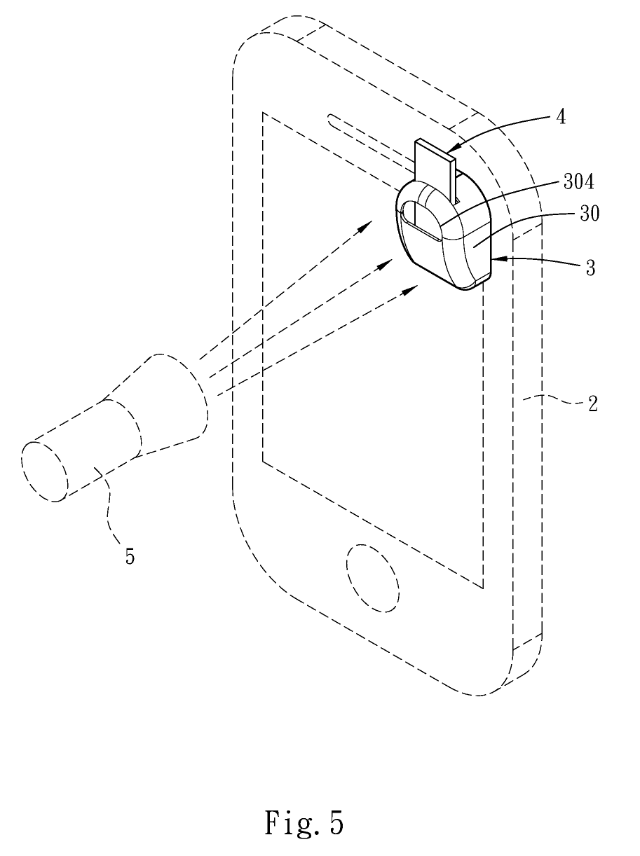

In the present invention, when the test area 40 and the calibration area 41 at one end of the biochemical sheet 4 are accommodated in the accommodating space 300, depending on actual conditions, the electronic computing device 2 may include an application program (not shown) electrically connected to the image capturing unit 21. Through the application program, the electronic computing device 2 of the present invention divides one part of the display unit 20 into a light emitting area 200, and locates the light emitting area 200 below the opening 303 of the main body 30 to have the illumination provided by the light emitting area 200 to serve as a main light source for the image capturing unit 21 to capture an image of the test area 40. Further, light beams from the light emitting area 200 irradiate into the accommodating space 300 through the opening 303 to provide the image capturing unit 21 with sufficient light beams for scanning and facilitating the observation for a change result of the test area 40. Alternatively, as shown in FIG. 5, depending on actual conditions, the main body 30 includes a through hole 304, which is located at one side of the main body 30 opposite the optical lens 302 and is in communication with the accommodating space 300. The user may additionally apply an external light source such as a torch 5 to utilize illumination of the external light source as the main light source for the image capturing unit 21 to capture an image of the test area 40. The light beams from the external light source irradiate into the accommodating space 300 through the through hole 304 to provide the image capturing unit 21 with more sufficient light beams for scanning a change result of the test area 40. Further, when the illumination of the light emitting area 200 serves as the main light source for the image capturing unit 21 to capture the change result of the test area 40, the external light source may also serve as an auxiliary light source for the image capturing unit 21 to capture the change result of the test area 40.

As previous described, in the present invention, the light emitting area 200 on the display unit 20 provides a lighting function. Further, through the control of the application program, the shape of the light emitting area 200 may be made to be consistent with the shape of the main body 30, so as to allow the optical lens 302 of the main body 30 to align with a center point of the image capturing unit 21.

Further, the application program may control a position of the display unit 20 to form a display area 201, which is located at a center part of the display unit 20. The electronic computing device 2 of the present invention may simultaneously divide the display unit 20 into the light emitting area 200 and the display area 201 through the application program, hence allowing the user to at the same time observe the change result of the test area 40 while the image capturing unit 21 scans the change result of the test area 40. Alternatively, through the control of the application program, the display unit 20 is caused to form the light emitting area 200 or the display area 201 at different time points, e.g., the display unit 20 activates only the light emitting area 200 during the detection, and activates the display area 201 to display the detection result after the detection is complete.

In one embodiment of the present invention, the main body 30 may be provided as a housing having better reflectivity. When light beams enter the accommodating space 300, the light beams are less likely absorbed by the main body 30. Thus, more light beams can be reflected to the test area 40 and the calibration area 41, such that the image capturing unit 21 is provided with more sufficient light beams for scanning the change result of the object under test mixed with the biochemical reactant on the biochemical sheet 4.

The calibration area 41 includes at least one comparison reference object (not shown). The comparison reference object is comparison reference data that is embedded in the calibration area 41 according to the type of the object under test to be detected. More specifically, to detect an amount of a metal component or a pH value, corresponding data such a comparison value or a form is embedded into the calibration area 41 to serve as a comparison standard for the current detection. To detect blood sugar in blood, urine or saliva, corresponding data is similarly embedded into the calibration area 41, and so forth.

The present invention is suitable for various types of electronic computing devices. However, these electronic computing devices in different brands or models may have different screen display or brightness parameter settings, or numerous data settings (e.g. color temperature and white balance) of the image capturing unit 21 may also be different. Further, assuming that the image capturing unit 21 adopts non-automatic focusing and the precision levels by which the user operates and inserts the biochemical sheet 4 are different, undesired effects of image recognition and determination errors may be resulted if preceding calibration operations are not provided before the application of the biochemical sheet 4. Therefore, the calibration area 41 of the present invention further includes at least one focusing target object (not shown), which is embedded into the calibration area 41. Before the biochemical sheet 4 is inserted, calibration operations including insertion alignment, image focusing and white balance are first performed for the image capturing unit 21 of the electronic computing device 2 to enhance the detection accuracy. In one embodiment, the focusing target object may be implemented as a calibration object in form of a plurality of thick/thin strips or grids for aligning an object or a light source or calibrating white balance.

As previously described, one end of the biochemical sheet 4 is simultaneously embedded with the test area 40 and the calibration area 41, so that the user may directly compare the change result of the test area 40 with the comparison reference object at the calibration area 41, which is distinct from the drawback of the test area 40 and the calibration area 41 belonging to two different objects and being more costly and inconvenient of a conventional solution. It should be noted that, the comparison reference object or the focusing target object may be stored in the electronic computing device 2. Further, the focusing target object may be an identification password of the manufacturer of the biochemical sheet 4, hence allowing only predetermined biochemical sheets 4 to be activated and used for detection by the electronic computing device 2.

Further, when detection is performed using the biochemical sheet 4, the change result of the test area 40 can be manually observed. Alternatively, the image capturing unit 21 is controlled by the application program to cause the image capturing unit 21 to automatically scan the change result of the test area 40 and the comparison reference object. A difference between the change result of the test area 40 and the comparison reference object is then automatically calculated, and the calculated result is displayed on the display area 201 of the display unit 20.

Further, the electronic computing device 2 includes a database (not shown) electrically connected to the application program. The calculated result of the application program may be stored in the database to serve for data statistics and data analysis purposes, or to serve for subsequent support applications after a connection is established with a remote server.

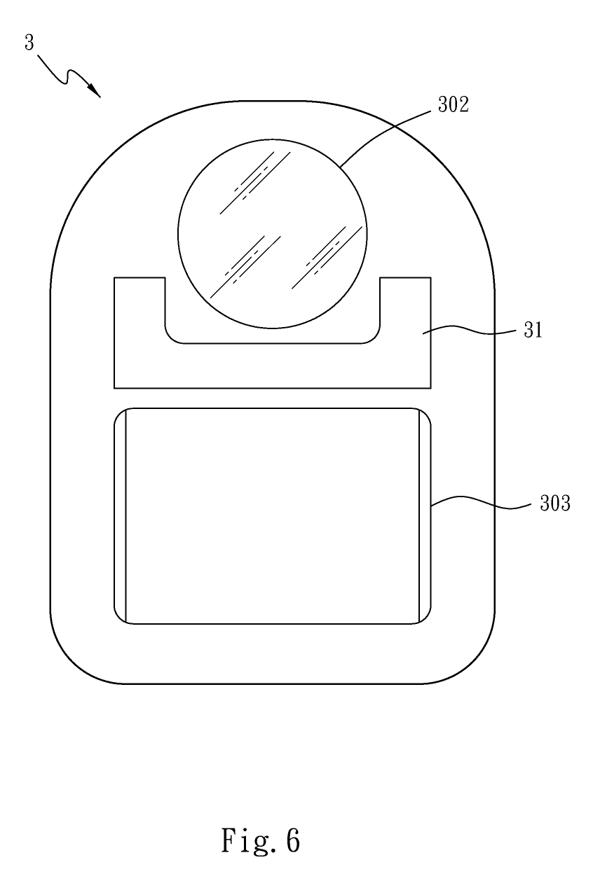

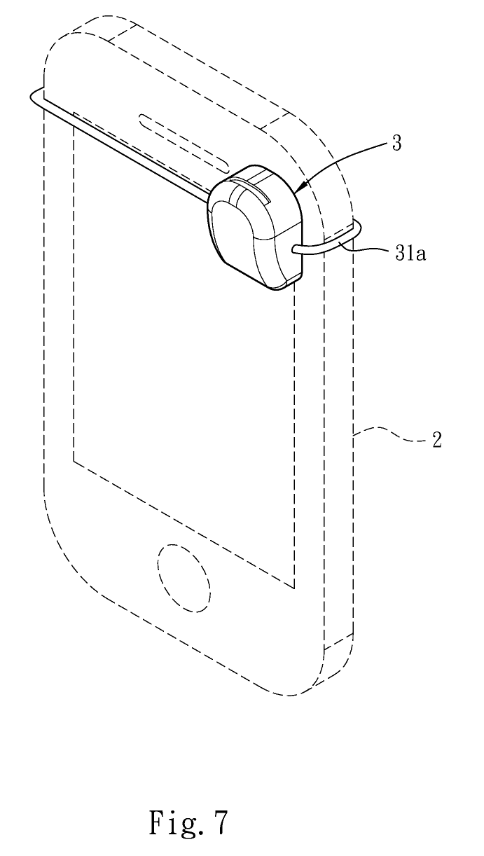

Referring to FIG. 6 and FIG. 7, the optical device 3 includes a fixing member for fixing the main body 30 on the electronic computing device 2. The fixing member is coplanar with the optical lens 302 and the opening 303 of the main body 30, and is located between the optical lens 302 and the opening 303. The fixing member may be implemented by an adhesive film 31, a rubber band 31a, or a clamping tool (not shown). In the present invention, the optical device 3 is fixed on the electronic computing device 2 using the fixing member, so as to prevent the electronic computing device 2 or the optical device 3 from human impacts that may dislocate the electronic computing device 2 or the optical device 3.

What is claimed is:

1. A biochemical detection apparatus, comprising:
an electronic computing device, comprising at least one display unit, and at least one image capturing unit coplanar with the display unit; and
an optical device, comprising a main body, the main body comprising an accommodating space provided in the main body, an insertion slot in communication with the accommodating space, an optical lens in communication with the accommodating space, and an opening in communication with the accommodating space;
wherein, the optical lens and the opening are disposed on same plane of the main body, and the optical lens is located above the image capturing unit.

2. The biochemical detection apparatus of claim 1, wherein the main body comprises a through hole located at one side of the main body opposite the optical lens and being in communication with the accommodating space.

3. The biochemical detection apparatus of claim 1, wherein the optical device comprises a fixing member for fixing the main body on the electronic computing device.

4. The biochemical detection apparatus of claim 1, further comprising:
a biochemical sheet, inserted in the insertion slot, comprising a test area and a calibration area at one end thereof, the test area and the calibration area being located above the image capturing unit.

5. The biochemical detection apparatus of claim 4, wherein the electronic computing device comprises an application program electrically connected to the image capturing unit.

6. The biochemical detection apparatus of claim 5, wherein the application program controls a part of the display unit near the image capturing unit to form a light emitting area, and the opening of the main body is located above the light emitting area.

7. The biochemical detection apparatus of claim 5, wherein the application program controls a position of the display unit to form a display area.

8. The biochemical detection apparatus of claim 5, wherein the electronic computing device comprises a database electrically connected to the application program.

9. The biochemical detection apparatus of claim 4, wherein the calibration area comprises at least one comparison reference object.

10. The biochemical detection apparatus of claim 4, wherein the calibration area comprises at least one focusing target object.
